(19) United States
(12) Patent Application Publication
(10) Pub. No.: US 2022/0117075 A1
(43) Pub. Date: Apr. 14, 2022

(54) SYSTEMS AND METHODS FOR COMPACT LASER WAKEFIELD ACCELERATED ELECTRONS AND X-RAYS

(71) Applicants: THE REGENTS OF THE UNIVERSITY OF CALIFORNIA, Oakland, CA (US); TAE TECHNOLOGIES, INC., Foothill Ranch, CA (US)

(72) Inventors: Toshiki Tajima, Foothill Ranch, CA (US); Gerard Mourou, Paris (FR); Dante Roa, Mission Viejo, CA (US); Ales Necas, Greensboro, NC (US)

(21) Appl. No.: 17/476,569

(22) Filed: Sep. 16, 2021

Related U.S. Application Data
(63) Continuation of application No. PCT/US20/23394, filed on Mar. 18, 2020.
(60) Provisional application No. 62/819,918, filed on Mar. 18, 2019.

(51) Int. Cl. H05H 15/00; A61N 5/10; H05G 2/00

(52) U.S. Cl. CPC H05H 15/00; A61N 5/10; A61N 2005/1088; H05G 2/008; H05G 2/003

(57) ABSTRACT

A laser wakefield acceleration (LWFA) induced electron beam system for cancer therapy and diagnostics. Example embodiments presented herein include one or more laser fibers, and an electron beam source within an individual one of the one or more laser fibers, wherein the electron beam source includes a laser pulse source, a plasma target, a set of optics interposing the laser pulse source and the plasma target adapted to focus a laser pulse generated by the laser pulse source onto the plasma target, wherein interaction of the laser pulse with the plasma target induces the generation of an electron beam. In various embodiments presented herein, high energy electrons of the electron beam interact with a high-Z material to generate X-rays.

### CROSS-REFERENCE TO RELATED APPLICATIONS

The subject application is a continuation of International Patent Application No. PCT/US20/23394, filed Mar. 18, 2020, which claims priority to U.S. Provisional Patent Application No. 62/819,918, filed on Mar. 18, 2019, both of which are incorporated by reference herein in their entireties for all purposes.

### FIELD

The subject matter described herein relates generally to laser wakefield acceleration (LWFA) and, more particularly, to systems and methods that facilitate the generation of a large dose electron beam or X-ray produced compactly by LWFA and, more particularly, to systems and methods that facilitate medical treatments and diagnostics for cancer and the like with electron beams and X-rays, and that facilitate irradiation of instruments and materials with electron beams for surface sterilization.

### BACKGROUND

The use of radiation in medicine dates back to more than a century and its applications have been in diagnostic imaging and radiation therapy [see, Barret et al., Radiological Imaging: The theory of image formation, detection and processing. Vols. 1 and 2, Academic Press, 1981; Johns et al., The physics of radiology, 3^{rd}, 1974]. For diagnostic imaging, kilovolt (KV) X-ray beams produced by the collision of fast-moving electrons with a tungsten target have been the standard technology for many years that continues to this day [see, Beutel et al., Handbook of Medical Imaging, Vol 1, SPIE Press, 2000; Curry et al., Christensen's Physics of diagnostic radiology, 4^{th} Ed., 1990]. All radiological imaging systems like radiography, mammography, fluoroscopy and computer tomography produce their imaging X-rays via this technology. Production of KV X-rays through this technology has proven effective, however, there can be a significant benefit if KV X-ray beams can be generated with a more compact device that can make some of the existing imaging apparatuses less bulky and therefore, less intimidating to a patient. Many of the treatments use radioisotopes for irradiation; the accompanying logistics of radioisotopes production, transportation and storage is a major reason for looking into different sources. For example, all radioisotopes have a characteristics half-life, therefore, if not timely used, it will be lost. Moreover, all radioisotopes are covered under the export-control laws and are heavily guarded against proliferation.

Radiation therapy [see, Khan, The physics of radiation therapy, 4^{th} Ed., 2010], which focuses primarily on treating cancer, has benefitted significantly from various radiation sources. Megavolt (MV) X-ray and (MeV) electron beams generated by a linear accelerator (linac) are routinely used to treat cancerous tumors in any part of the body. Production of these beams are based on a similar concept as the KV X-rays for imaging with the exception that the electrons are accelerated to megavoltage energies by an electric field component of a radiofrequency (RF) source. The waveguide, where the electron acceleration occurs, can be more than a meter long while the RF source can be just as big. A significant innovation can be envisioned if production of MV X-ray and MeV electron beams can be achieved within a fraction of the size of current linacs and with the same beam characteristics. The use a compact laser wakefield acceleration (LWFA) [see, Tajima et al., “Laser electron accelerator”, Phys. Rev. Ltrs. 43.4 (1979), 267] based on the Coherent Amplification Network (CAN) [see, Mourou et al., “The future is fibre accelerators”, Nature Photonics 7, 258-261 (2013)] revolutionized the production of low energy/ultra-high dose electrons and high energy electrons by making it more cost effective and more accessible to more radiation oncology centers.

Brachytherapy is another treatment technique within radiation oncology that delivers a radiation dose to adjacent and/or in close proximity to a target volume. Historically, radioactive sources like Ra-222, Ir-192, Co-60 among others have been used in brachytherapy. High-dose-rate (HDR) brachytherapy [see, Kubo et al., “High dose-rate brachytherapy treatment delivery: report of the AAPM Radiation Therapy Committee Task Group”, 59, Med. Phys. 25: 375-403, 1998] utilizes a high activity (10 Ci) radioactive gamma-ray source to treat gynecological, breast, skin and head-and-neck cancers among others, since it can deliver a very conformal dose to a target and minimize dose to nearby organs and regions beyond the target location. Although, the use of a radioactive source in a HDR treatment is effective, a treatment can take progressively longer times due to source decay. For instance, a HDR gynecological treatment with a brand new Ir-192 source (10 Ci) can take a little over 5 min compared to 15 min with a source that is four months old. Significant benefits can be realized by replacing a radioactive source in HDR treatments for an electronically generated X-ray and/or electron beam such as eliminating regular source replacement due to decay, reduction in radiation shielding and constant treatment times.

Surgical instruments along with other components and material require sterilization. The death of biologically active organisms (viruses, bacteria, micro-organisms) on surfaces is important to sterilization. Conventional methods of sterilization of instruments, components and materials include, among other things, steam (autoclave) sterilization, gas (ethylene oxide) sterilization, and dry heat sterilization using a glass bead sterilizer. The disadvantages associated with each method range from harm to instruments, components or material, to harm to personnel.

For these and other reasons, needs exist for improved systems, devices, and methods for energy systems for medical treatments and diagnostics as well as for sterilization methods.

### SUMMARY

Example embodiments of systems, devices, and methods are provided herein to facilitate the generation of low-intensity laser, electron beam and X-rays for medical treatments and theranostics including, e.g., treating cancer and cancer theranostics, as well as for the sterilization of surgical instruments and other components and materials.

In example embodiments, laser wakefield acceleration (LWFA) is used to generate electron beams or X-rays to facilitate medical treatments or therapies, such as, e.g., irradiation of cancer or tumors. A high dose of electrons or X-rays is achieved as a result of a combination of effects including a plurality of fiber lasers, a low energy (high plasma density) regime of laser wakefield acceleration, a high energy (low plasma density) regime of laser wakefield acceleration, a high repetition rate of the laser, and a targeting of the tumor at a closer distance and smaller volume, and an optimal shaping of the fibers to match the shape of the delivery of the required dose of electrons or X-rays to the shape of the tumor while maintaining healthy tissue intact.

In further example embodiments, the diagnostics and treatment progress monitoring is performed via emission, such as, e.g., fluorescence induced by low intensity laser, X-rays, or electron beam.

In further example embodiments, two (2) operational regimes are formed: (1) a low energy/ultra-high dose electron beam (˜1 MeV) originating from an interaction of a laser with a high density plasma (10²⁰˜10²¹ electrons/cm³); and, (2) a high energy electron beam (1-20 MeV) originating from an interaction of a laser with a low density plasma (10¹⁸-10¹⁹ electrons/cm³).

In further example embodiments, the low energy/ultra-high dose electron beam is used for therapies, such as, e.g., irradiation of cancer or tumors.

In further example embodiments, the low-intensity laser is used for diagnostics via laser-induced fluorescence.

In further example embodiments, the low energy/variable dose electron beam is used for the diagnostics.

In further example embodiments, the high energy/variable dose electron beam is used for therapies or treatments, diagnostics and generation of X-rays.

In further example embodiments, the X-rays are formed by an interaction of the high energy electron beam with a high-Z material located at a tip of the laser fiber.

In further example embodiments, targeted cancer therapy or treatment and diagnostics are performed with X-rays generated by an electron beam impinging on nanoparticles located in or next to cancer or tumor cells and carrying a high-Z material.

In further example embodiments, the X-rays are used for cancer therapy or treatment and diagnostics via, e.g., X-rays induce fluorescence.

In various embodiments provided herein, the laser electron beam or X-ray are to be deployed or delivered, for example, via endoscopy, brachytherapy, or intra-operative radiation therapy (TORT).

In various embodiments provided herein, therapy and diagnostics are performed in real-time with feedback and controlled via an artificial neural network (ANN).

In various embodiments provided herein lenses, OPCPA [see, Budriūnas et al., “53 W average power CEP-stabilized OPCPA system delivering 55 TW few cycle pulses at 1 kHz repetition rate,” Opt. Express 25, 5797 (2017)] or CPA [see, Strickland et al., “Compression of amplified chirped optical pulses,” Opt. Commun. 56, 219-221 (1985)] are used to compress CAN or fiber laser.

In further example embodiments, the laser architecture is configured to deliver 10's of fs pulses of milli-joule energies. When longer pulses (i.e. non-resonant LWFA) are adopted, either due to a longer pulse length or higher electron density, the exciting of wakefields by way of self-modulated LWFA (i.e. SMLWFA) or an appropriate superposition of laser pulses are adopted to induce appropriate wakefields (the beat waves, or pulse superpositions).

In further example embodiments, the laser intensity is in the range 10¹⁷ W/cm² to 10¹⁹ W/cm².

In further example embodiments, the laser adopts a high repetition rate that is greater than 100,000 Hz.

In various embodiments provided herein, CAN laser fibers are micrometric. Thus it may be easily carried by either a surgeon or a robot externally or internally. Internal bodily applications may include accessing the body interior from a bodily opening and via veins. An example of this application can be the treatment of liver tumors [see, Arnold et al., “90Y-TheraSpheres: The new look of Yttrium-90,” Am. J. Surg. Pathol. 43: 688-694, 2019], where an interventional radiologist inserts a micro-catheter through a patient's femoral artery near the groin. This catheter is guided to the hepatic artery from which the tumor gets most of its blood supply and therefore provides an effective conduit for irradiating the tumor. CAN laser fibers could be inserted through the micro-catheter and guided to the tumor via the tumor's blood supply to provide the treatment.

In further example embodiments, fibers (CAN or fiber laser) are shaped and modified to conform the shape of the dose and diagnostics to the shape of the tumor while maintaining healthy tissue intact.

Cancer treatment based upon CAN fiber technology along with low and high density targets to accelerate electrons allows for a fine control of the electron energy thus targeting the tumor preferentially. Furthermore, by using a plurality of fibers to deliver a dose of electrons or X-rays, conforming the shape of the delivered dose to any arbitrary tumor shape can be controlled as well.

In further example embodiments, LWFA electron beams are used for sterilization of instruments, components and material surfaces. Irradiation of the surfaces of instruments, components and material with electron beams and X-rays causes cell apoptosis—i.e., pre-programmed cell death. The death of biologically active organisms (viruses, bacteria, micro-organisms) on surfaces is important to sterilization.

Advantages of the example embodiments of laser generated electrons include:

a) Small size of the laser-driven electron beams and their targets.

b) Fine electron control: temporal as well as spatial.

c) High repetition rate of the lasers

d) High laser wall plug efficiency of 30%.

Other systems, devices, methods, features and advantages of the subject matter described herein will be or will become apparent to one with skill in the art upon examination of the following figures and detailed description. It is intended that all such additional systems, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a schematic of an example embodiment illustrating the generation of electrons by lasers. FIG. 1 further illustrates the generation of X-rays within a tumor.

FIG. 2 is a schematic of an example embodiment illustrating the generation of electrons by lasers. FIG. 2 further illustrates the generation of X-rays by electron interaction with high-Z material.

FIGS. 3A and 3B are schematics illustrates an example embodiment of laser fibers.

FIG. 4 is a schematic of an example embodiment illustrating a laser source and laser fiber delivery to a patient.

FIG. 5 is a schematic of an example of a conventional system for the generation and amplification of a laser pulse.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

Example embodiments of laser wakefield acceleration (LWFA) based electron beam or X-ray systems are described herein, as are: example embodiments of devices and components within such systems; example embodiments of methods of operating and using such systems; and example embodiments of applications in which such systems can be implemented or incorporated or with which such systems can be utilized.

Each of the additional features and teachings disclosed below can be utilized separately or in conjunction with other features and teachings to provide systems and methods that facilitate high dose irradiation by an electron beam generated via LWFA and delivered to the tumor by a high repetition rate CAN laser system as well as laser based theranostics.

In various example embodiments provided herein, a laser fiber is understood as either a single fiber or the coherent network of fibers—known as a Coherent Amplified Network (CAN).

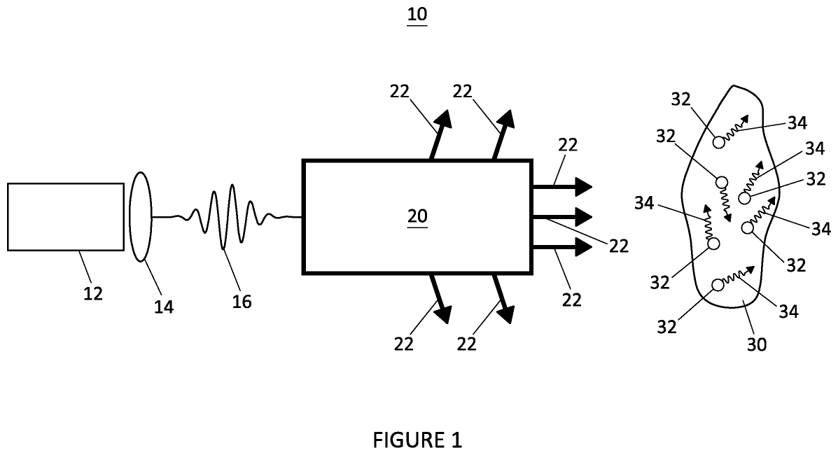

Turning to figures, FIG. 1 shows an example embodiment of an assembly comprising electron and X-ray sources. The assembly includes a laser fiber 12, optics 14 optically coupled to the laser fiber 12, and a supply of a precursor to a plasma 20 such as, e.g., a neutral gas, including, e.g., nitrogen, helium or the like, or carbon nanotubes or nano-particles. The laser fiber 12 delivers a long pulse, which is used to generate an electron beam, X-rays and laser induced fluorescents, to a set of optics 14 that focuses the laser pulse in space.

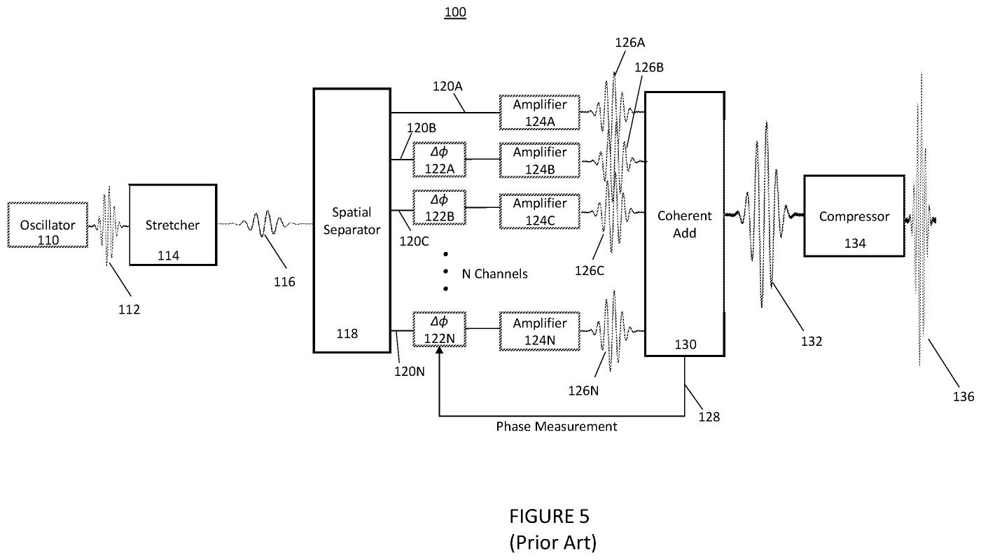

Turning to FIG. 5, one example of conventional methods for generating and amplifying an appropriate laser pulse is shown and provided for example purposes only. To generate an appropriate laser pulse, a laser 100 includes an oscillator 110. The oscillator 110 creates a laser pulse 112, such as, e.g., a nano-joule, femtosecond laser pulse. The pulse energy of the laser pulse 112 is amplified based on the chirped-pulse-amplification (CPA) principle. First the laser pulse 112 is stretched by a stretcher 114, such as, e.g., a Chirped Fiber Bragg Grating (CFBG) stretcher, so that a chirped laser pulse 116, such as, e.g., a laser pulse stretched to nanoseconds, becomes positively chirped with the long wavelength preceding the shorter wavelengths. Next the chirped laser pulse 116 is spatially separated by a spatial separator 118 into N amplification channels 120A, 120B, 120C . . . 120N. Before amplification the relative phase and delay of each channel Δϕ 122A, 122B . . . 122N is then controlled relative to a reference pulse based on the phase measurement feedback 128 from a monitor 130 of the coherent addition stage. The delay between the channels 120A, 120B, 120C . . . 120N is managed by using a variable optical delay line while the phase difference is controlled by a fiber stretcher 114 that physically stretches a section of fiber. The amplification of the N pulses takes place within N amplifiers 124A, 124B, 124C . . . 124N having photonic crystal fibers (PCF) doped with a rare earth material, such as, e.g., ytterbium. Then the amplified pulses 126A, 126B, 126C . . . 126N are coherently added by a coherent add lens 130 focusing a hexagonal array of the N pulses exiting the fibers arranged within a precision mount. The amplified, recombined pulse 132 is still positively chirped and is sent to a conventional grating-based compressor 134 that reverses the dispersion of the stretcher to generate an ultra-short laser pulse 136 such as, e.g., femtosecond, milli-joule or joule energy level pulse. The ultra-short laser pulse 136 can be delivered to a cancer or tumor site via fibers to irradiate targets.

Returning back to FIG. 1, the set of optics 14 focuses a compressed pulse 16 onto the precursor to the plasma 20. Either a separate low intensity laser pulse delivered from the laser fiber 12 or the pedestal of the main pulse delivered from the laser fiber 12 ionizes the neutral gas to form a lower-than-gas density plasma 20 (10¹⁸-10¹⁹ electrons/cm³). The laser-plasma interaction consequently generates high energy electrons 22. The electrons 22 can be used to directly irradiate a tumor 30.

When a laser pulse interacts with a low density target (nₑ«n_{c}) only a few electrons are captured in the laser wake generating a low flux of high energy electrons. in a manner analogous to a tsunami wave propagating in a deep ocean; it does not couple well to objects since the tsunami's phase velocity is too big. However, once the tsunami comes to the shore or shallow water, its phase velocity decreases and coupling to even stationary objects is possible while the amplitude increases. Similarly, when a laser interacts with a high-density plasma (nₑ≈n_{c}), the laser's phase velocity reduces and strong coupling to the plasma occurs at the expense the average electron energy is lower, but still on the order of 100s keV. However, the flux, and therefore the dose, is large. The target is specially designed to meet nₑ≈n_{c} conditions. This may be achievable using optimally packed carbon nanotubes or nano particles.

In further example embodiments, the electrons 22 interact with nanoparticles 32 carrying a high-Z material, such as, e.g., gold or gadolinium, which generates X-rays 34 that irradiate the tumor 30. Although the laser generated electrons 22 can interact with cancer or tumor cells causing a cell death—apoptosis, electron interaction with cancer or tumor cells can be enhanced (1000×) and electron energy delivery can be predominantly localized to the cancer or tumor volume by impregnating the cancer or tumor volume with high-Z material such as, e.g., gold or gadolinium. The tumor 30 may be impregnated with the high-Z material carrying nanoparticles 32 via different delivery strategies such as, e.g., topical (e.g., as an ointment), needle injection or vector drug delivery. When an electron interacts with a high-Z material, its energy is converted to a X-ray photon 34 through the process of Bremsstrahlung. The high-Z material carried by the nanoparticles 32 preferentially slow down the electrons 22 within the cancerous mass or tumor 30 and convert a portion of the electron energy to photons 34. The photons 34 generated by converting the electron energy are consequently absorbed by the surrounding cancer or tumor cells causing the cancer or tumor cell death.

In additional example embodiments of FIG. 1, instead of ionizing a neutral gas, the plasma 20 is formed by ionizing a carbon nanotube foam to form a near-critical density electron plasma (10²⁰˜10²¹ electrons/cm³) to generate an ultra-high dose of low energy (˜1 MeV) electrons 22 to irradiate the tumor 30. In this embodiment, the electrons 22 are not energetic enough to cause sufficient amount of X-rays. The ionizing of the carbon nanotube foam 33 is performed by a pedestal of the main laser pulse or a separate low-intensity laser pulse from the fiber laser 12.

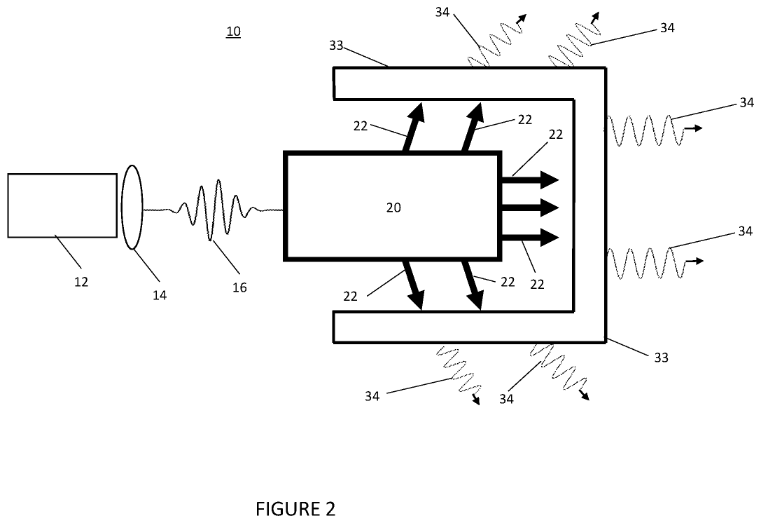

In another example embodiment shown in FIG. 2, the assembly includes a high-Z material 33 positioned about the neutral gas 20. The X-rays 34 are produced by the interaction of the high energy electrons 32 with the high-Z material 33. The electrons 22 are generated from a low density plasma 20.

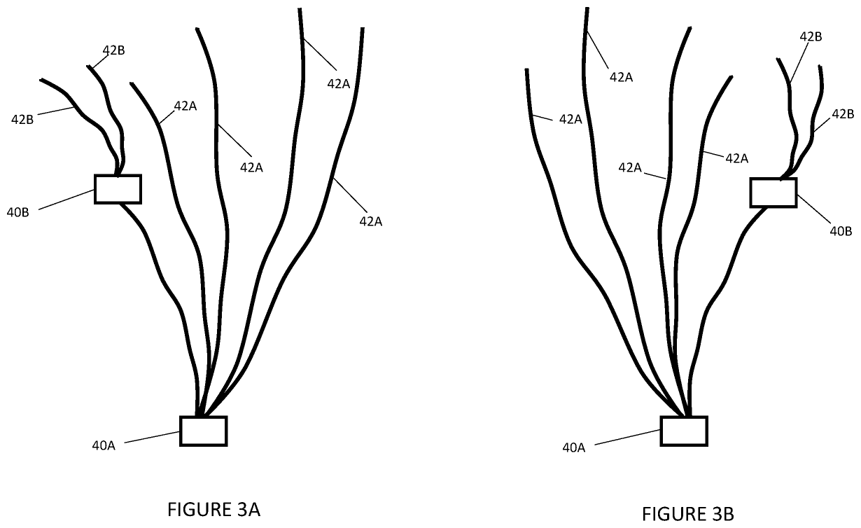

Turning to FIGS. 3A and 3B, an example representation of fiber lasers 42A and 42B originating from splitters 40A and 40B, are shown (laser source is not shown). The shape of the fiber configuration is optimized to the delivery of a required dose of electrons or X-rays preferentially to the tumor while minimizing irradiation of the healthy surrounding tissue and eliminating the need for dwell time. The fibers are inserted into a patient via a flexible catheter for treatment of, e.g., liver cancer, or a rigid channel for treatment of, e.g. ovarian cancer. The fibers are insertable via a vein or artery as well.

As further shown in FIGS. 3A and 3B, a single fiber laser may be further split by a second splitter 40B to further conform the dose localization and dose shaping.

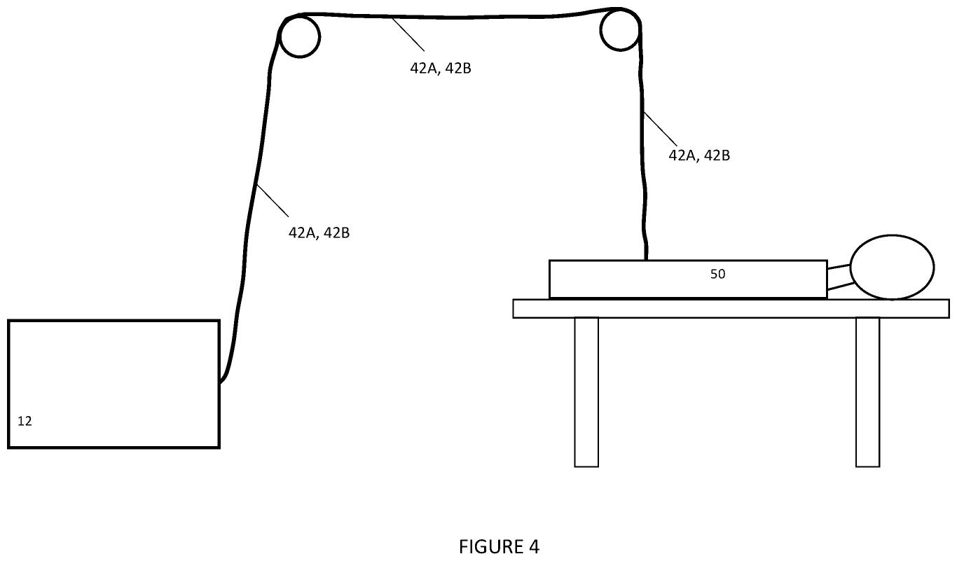

Turning to FIG. 4, an example embodiment is shown to include a laser source 12 and a fiber 42A, 42B. The fiber 42A, 42B delivers the laser pulses to the patient 50. The end of fiber 42A, 42B enters the patient 50 or is used during the intra-operative radiation therapy (IORT). The end of fiber 42A, 42B is shaped as shown in FIGS. 3A and 3B and the tip of each fiber contains an electron beam source 20 as shown in FIGS. 1 and 2 with added potential for X-ray 22 generation.

In further example embodiments, two (2) operational regimes are formed: (1) a low energy/ultra-high dose electron beam (˜1 MeV) originating from an interaction of a laser with a high density plasma (10²⁰˜10²¹ electrons/cm³); and, (2) a high energy electron beam (1-20 MeV) originating from an interaction of a laser with a low density plasma (10¹⁸-10¹⁹ electrons/cm³).

In further example embodiments, the low energy/ultra-high dose electron beam is used for therapies, such as, e.g., irradiation of cancer or tumors.

In further example embodiments, the low-intensity laser is used for diagnostics via laser-induced fluorescence.

In further example embodiments, the low energy/variable dose electron beam is used for the diagnostics.

In further example embodiments, the high energy/variable dose electron beam is used for therapies or treatments, diagnostics and generation of X-rays.

In further example embodiments, the X-rays are formed by an interaction of the high energy electron beam with a high-Z material located at a tip of the laser fiber.

In further example embodiments, targeted cancer therapy or treatment and diagnostics are performed with X-rays generated by an electron beam impinging on nanoparticles located in or next to cancer or tumor cells and carrying a high-Z material.

In further example embodiments, the X-rays are used for cancer therapy or treatment and diagnostics via, e.g., X-rays induce fluorescence.

In various embodiments provided herein, the laser electron beam or X-ray are to be deployed or delivered, for example, via endoscopy, brachytherapy, or intra-operative radiation therapy (IORT).

In various embodiments provided herein, therapy and diagnostics are performed in real-time with feedback and controlled via an artificial neural network (ANN).

In various embodiments provided herein lenses, OPCPA [see, Budriūnas et al., 25, 5797 (2017)] or CPA [see, Strickland et al., 56, 219-221 (1985)] are used to compress CAN or fiber laser.

In further example embodiments, the laser architecture is configured to deliver 10's of fs pulses of milli-joule energies. When longer pulses (i.e. non-resonant LWFA) are adopted, either due to a longer pulse length or higher electron density, the exciting of wakefields by way of self-modulated LWFA (i.e. SMLWFA) or an appropriate superposition of laser pulses are adopted to induce appropriate wakefields (the beat waves, or pulse superpositions).

In further example embodiments, the laser intensity is in the range 10¹⁷W/cm² to 10¹⁹ W/cm².

In further example embodiments, the laser adopts a high repetition rate that is greater than 100,000 Hz.

In various embodiments provided herein, CAN laser fibers are micrometric. Thus it may be easily carried by either a surgeon or a robot externally or internally. Internal bodily applications may include accessing the body interior from a bodily opening and via veins. An example of this application can be the treatment of liver tumors [see, Arnold et al., Am. J. Surg. Pathol. 43: 688-694, 2019], where an interventional radiologist inserts a micro-catheter through a patient's femoral artery near the groin. This catheter is guided to the hepatic artery from which the tumor gets most of its blood supply and therefore provides an effective conduit for irradiating the tumor. CAN laser fibers could be inserted through the micro-catheter and guided to the tumor via the tumor's blood supply to provide the treatment.

In further example embodiments, fibers (CAN or fiber laser) are shaped and modified to conform the shape of the dose and diagnostics to the shape of the tumor while maintaining healthy tissue intact.

Cancer treatment based upon CAN fiber technology along with low and high density targets to accelerate electrons allows for a fine control of the electron energy thus targeting the tumor preferentially. Furthermore, by using a plurality of fibers to deliver a dose of electrons or X-rays, conforming the shape of the delivered dose to any arbitrary tumor shape can be controlled as well.

In further example embodiments, LWFA electron beams are used for sterilization of instruments, components and material surfaces. Irradiation of the surfaces of instruments, components and material with electron beams and X-rays causes cell apoptosis—i.e., pre-programmed cell death. The death of biologically active organisms (viruses, bacteria, micro-organisms) on surfaces is important to sterilization.

Furthermore, in all example embodiments provided herein a diagnostics based on a low intensity laser, low/high energy electron beam, or X-ray is provided feedback from an artificial neural network system to optimize treatment and to study treatment progress.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

As used herein and in the appended claims, the singular forms “a,” “an,” and “the” include plural referents unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

What is claimed:

1. A laser wakefield acceleration (LWFA) induced electron beam system for cancer therapy and diagnostics comprising:
one or more laser fibers, and
an electron beam source within an individual one of the one or more laser fibers, wherein the electron beam source includes,
a laser pulse source,
a plasma target,
a set of optics interposing the laser pulse source and the plasma target adapted to focus a laser pulse generated by the laser pulse source onto the plasma target, wherein interaction of the laser pulse with the plasma target induces the generation of an electron beam.

2. The electron beam system of claim 1, wherein the one or more fibers includes one or more splitters.

3. The electron beam system of claim 2, wherein an end of the one or more fibers is configured to enter the patient or configured for intra-operative radiation therapy (IORT).

4. The electron beam system of claim 2, wherein an end of the one or more fibers comprises a tip having an electron beam source.

5. The electron beam system of claim 4, wherein electron beam source is configured for X-ray generation.

6. The electron beam system of claim 2, wherein the ends of a plurality of the one or more fiber are configurable to a shape of a target tumor.

7. The electron beam system of claim 2, wherein individual ones of the one or more fibers are insertable into a patient via one of a flexible catheter or a rigid channel.

8. The electron beam system of claim 4, wherein the laser pulse source is configurable to compress a pulse in time.

9. The electron beam system of claim 1, wherein the electron beam source is configured to utilize one of a separate low intensity laser pulse or a pedestal of a main laser pulse to ionizes a neutral gas into a lower-than-gas density plasma as the plasma target.

10. The electron beam system of claim 9, wherein a laser pulse generated by the laser pulse source interacts with the plasma target to generate high energy electrons.

11. The electron beam system of claim 10, further comprising a high-Z material positioned about the plasma target, wherein the high energy electrons interact with the high-Z material to generate X-rays.

12. The electron beam system of claim 9, wherein the plasma density is in a range of 10¹⁸-10¹⁹ electrons/cm³.

13. The electron beam system of claim 1, further comprising a monitoring system configured to monitor low intensity laser, X-ray or electron beam induced emissions.

14. The electron beam system of claim 1, wherein the electron beam system is configured to generate a low energy/ultra-high dose electron beam from an interaction of a laser pulse with a plasma having density in a range of 10²⁰˜10²¹electrons/cm³ or a high energy electron beam from an interaction of a laser with a plasma having a density in a range of 10¹⁸-10¹⁹ electrons/cm³.

15. The electron beam system of claim 1, further comprising one of an OPCPA lense or a CPA lense.

16. The electron beam system of claim 1, wherein the laser pulse source comprising a coherent amplified network.
